(19) United States
(12) Patent Application Publication
(10) Pub. No.: US 2016/0091373 A1
(43) Pub. Date: Mar. 31, 2016

(54) METHOD TO DETERMINE HEAT TRANSFER EFFICIENCY OF A HEATING DEVICE AND SYSTEM THEREFOR

(71) Applicant: SMITHS MEDICAL ASD, INC., Rockland, MA (US)

(72) Inventors: Paul Hattan, Minnetonka, MN (US); Christopher Kim Thorp, Minneapolis, MN (US); Robert Taylor Parsons, JR., Braham, MN (US); Jay Ellingboe, New Brighton, MN (US); Kristin Finberg, Minneapolis, MN (US); Laura-Lee Brown, Maplewood, MN (US); Chris Zander, Hudson, WI (US)

(73) Assignee: SMITHS MEDICAL ASD, INC., Rockland, MA (US)

(21) Appl. No.: 14/499,662

(22) Filed: Sep. 29, 2014

(51) Int. Cl. G01K 3/10; G01F 1/00; G01L 19/00; A61F 7/08; G01K 13/02

(52) U.S. Cl. CPC G01K 3/10; A61F 7/08; G01K 13/02; G01L 19/00; G01F 1/00; A61F 2007/006

(57) ABSTRACT

To determine the heat output efficacy of a heating device such as a convective warming blanket, a conduit structure is constructed to have coiled metal tubings that form the head, torso and other non-moving parts of a person analog. Flexible tubings interconnect the metal tubings to establish a continuous conduit between an inlet and an outlet of the structure, so that a fluid having a particular heat capacity may flow uninterrupted through the conduit structure. Respective temperature sensors at the inlet and outlet measure the input and output temperatures of the fluid. Using the flow rate, the heat capacity and the respective temperatures of the fluid measured at the inlet and outlet, the heat output efficiency of the blanket may be calculated. A FLIR camera may be used to obtain an IR image to confirm the heat output efficiency of the blanket.

### FIELD OF THE INVENTION

The present invention relates to a method, and system therefor, that provides a quantitative evaluation of how much heat energy can be transferred from a heating device to an object, and particularly relates to the determination of the amount of heat output by a convective warming blanket to a person.

### BACKGROUND OF THE INVENTION

Heating devices for medical uses such as convective warming blankets are currently tested in accordance with the protocol set forth by the International Electrotechnical Commission (IEC), an organization for standardization, under the medical electrical equipment standards IEC 80601-2-35. According to the protocol set forth in the '35 standard, a test bed is planted with a plurality of calibrated temperature sensors on its surface where the convective blanket is to be placed. Each contact surface temperature sensor is made up of a thermocouple that is in intimate contact with a copper square. There are multiple temperature sensors spread all over the contact surface of the test bed. The blanket to be tested is placed on the contact surface of the bed, with the air holes or apertures of the blanket facing the surface of the bed. With the blanket inflated by heated air from an air warmer, the heated air is output from the air holes of the blanket. The temperature of the output heated air is measured by the various sensors so that an overall efficiency of the heat output from the blanket can be estimated from the different temperatures of output heat measured by the various sensors.

This procedure to determine the heat output efficiency of a convective warming blanket is cumbersome and not particularly accurate in that a blanket has to be correctly positioned onto the test bed so that heated air output from certain air holes can be measured by selective sensors. Oftentimes, to emulate in use conditions, the being tested blanket has to be weighted down at different areas. This includes putting weights on top of a mannequin that has been placed on top of the being tested blanket. Further, that the current test method being based on the '35 standard requires that the surface of the blanket that has the air holes would always be facing the contact surface of the test bed. For those instances where the to be tested blanket is an underbody blanket or a poncho blanket such as those being sold by the assignee of the instant invention, less than accurate measurement of the overall heat output of the blanket may result from using the test based on the '35 standard.

A method, and system therefor, to more accurately measure the efficacy of the heat output to a person by a heating device, for example one of the aforenoted convective warming blankets, without the cumbersome steps of finely adjusting the position of the blanket relative to the contact surface of the test bed, and then having to estimate the overall heat output efficiency of the blanket from the myriad temperatures measured from the different sensors, is therefore needed.

### SUMMARY OF THE PRESENT INVENTION

The inventive method effects a direct measurement of the amount of heat provided by a heating device to a person, represented by a person analog in the form of a mannequin. The mannequin is wrapped by metal tubings wherethrough a fluid with a known heat capacity passes. The metal tubings are wrapped around the torso and other non-moving parts of the mannequin. Plastic PVC tubings interconnect the metal tubings at the moving joints of the mannequin and the interior of the mannequin so that a continuous conduit formed by the metal and plastic tubings adapted to enable a fluid to flow therethrough uninterrupted is carried by the mannequin. An inlet is provided at one end of the conduit while an outlet is provided at the other end of the conduit. The fluid is input to the conduit by way of the inlet and output from the conduit via the outlet. A fluid reservoir that may be a part of a temperature regulating machine, for example a chiller, has an output connector connected to the inlet and an input connector connected to the outlet of the person analog conduit structure, so that a pump in the temperature regulating device can circulate the fluid along and through the conduit.

The mannequin wrapped with the coiled tubings, or simply the conduit structure, is positioned relative to a heating device, such as a convective warming blanket, so that the heated air output from the heating device is directed to the mannequin. As the metal tubings, which may be made of aluminum, have the characteristic of readily transferring heat, the heat from the heating device received by the conduit is readily transferred into the fluid that is being circulated along the conduit, and thereby represents the amount of heat that is being received by the person analog conduit structure.

A temperature sensor is provided at the inlet to measure the temperature of the fluid as it is being input into the conduit. Another sensor is provided at the outlet of the conduit to measure the temperature of the fluid as the fluid leaves the conduit. The fluid, as it circulates along the conduit, receives the heat directed to the conduit structure. The heat thus received is carried by the fluid as the fluid exits the conduit. Thus, there is a temperature difference of the fluid between when the fluid is input to the conduit and when the fluid exits the conduit. This temperature difference is obtained by comparing the respective temperatures as measured by the corresponding sensors provided at the inlet and the outlet of the conduit that wraps around the mannequin, i.e. the conduit structure.

The inventive method is able to determine the heating rate, which may also be referred to as units of watts or the amount of heat, received by the person analog from the heating device, which may be a convective warming blanket, by using the change in the temperature of the fluid between the inlet and the outlet of the conduit structure, i.e., the measured temperature difference, in conjunction with other readily available information. Such information may include the mass flow rate, or simply flow rate, of the fluid along the conduit. The flow rate of the fluid is controlled by the pump that circulates the fluid into and out of the conduit structure, and is monitored by a flow meter. Another piece of information readily available is the heat capacity of the fluid which is inherent with the fluid that is used, although it should be noted that the heat capacity of the fluid changes with the temperature of the fluid.

In addition to being able to obtain the heating rate of the heating device at any given time, the respective temperatures of the flowing fluid at the inlet and outlet of the conduit structure may be continuously monitored and measured so that the heating rate from the heating device, and therefore its heat output efficiency, can be continuously updated.

Separately from, or in addition to, the use of a conduit structure to directly measure the heat being output from a heating device, the heat output of a heating device such as a convective warming blanket may also be obtained by positioning a forward-looking infrared (FLIR) heat camera relative to the heating device, and possibly with the person analog placed thereon. The amount of heat detected by the FLIR camera is shown as an IR image that shows the reflective apparent temperature of the heating device due to the energy being emitted from the heat source. A guesstimate of the heat output efficiency of the heating device may be made by subtracting the energy reflected by the object from the energy emitted by the camera. The IR image from the FLIR camera may be used as a complement to the above discussed direct heat output measurement method.

The instant invention is therefore directed to a method of determining heat output efficiency of a heating device, comprising the steps of:

a) configuring a conduit to from a structure having an inlet and an outlet adapted to be positioned relative to the heating device;

b) circulating a fluid having a heat capacity along the conduit of the structure at a flow rate;

c) measuring the respective temperatures of the fluid at the inlet and the outlet of the structure; and

d) calculating a heating rate that is representative of the amount of heat received by the structure from the heating device by using the flow rate and the measured respective input and output temperatures of the fluid.

The instant invention is also directed to an apparatus for determining the heat output efficiency of a heating device that comprises: a structure formed by a conduit having an inlet and an outlet positioned relative to the heating device; a pump for circulating a fluid having a heat capacity along the conduit at a flow rate; respective sensors at the inlet and the outlet of the structure for measuring the corresponding temperatures of the fluid at the inlet and the outlet; and processor means for calculating a heating rate representative of the amount of heat received by the structure from the heating device by using the flow rate of the fluid and the measured inlet and outlet temperatures.

The instant invention is furthermore directed to an apparatus for determining the heat output efficiency of a convective blanket formed by two air impermeable layers inflatable by heated air and having selectively placed apertures at one of its layers. The inventive apparatus comprises: a structure formed by a conduit having an inlet and an outlet positioned relative to the blanket such that at least some of the apertures face the structure for outputting heated air towards the structure; a pump for circulating a fluid along the conduit at a particular flow rate; respective sensors at the inlet and the outlet of the structure for measuring the temperature of the fluid at the inlet and the temperature of the fluid at the outlet; and a processor for calculating a heating rate representative of the amount of heat received by the structure from the blanket by using the flow rate and the measured inlet and the outlet temperatures of the fluid.

### BRIEF DESCRIPTION OF THE FIGURES

The present invention will become apparent and the invention itself will be best understood with reference to the following description of the invention taken in conjunction with the accompanying drawings, wherein:

FIG. 1 is an illustration of an exemplar system for determining the heat output efficacy of a heating device;

FIG. 2 is an illustration of a patient or person analog of the instant invention, in the shape of a mannequin with tubings coiled thereabout to form a conduit structure;

FIG. 3 is another view of the person analog of FIG. 2 showing additional tubings and valves that connect to the inlet and outlet of the conduit structure;

FIG. 4 is an illustration showing a convective warming blanket placed over the person analog conduit structure of the instant invention;

FIG. 5 is a flowchart illustrating the steps for calculating the output heat efficiency, or heating rate, of a heating device embodied by a convective warming blanket;

FIG. 6 is another illustration of the system of the instant invention whereto is added a FLIR camera;

FIG. 7 shows the person analog conduit structure of FIG. 2 lying on a main portion of a convective blanket while having its front torso covered by anther portion of the convective warming blanket, as well as a display that shows an IR image; and

FIG. 8 is an illustration of the respective output heating rates, expressed in watts, of four different blankets, and the comparison of those directly measured output heating rates with corresponding IR images of the different blankets obtained by using a FLIR camera.

### DETAILED DESCRIPTION OF THE INVENTION

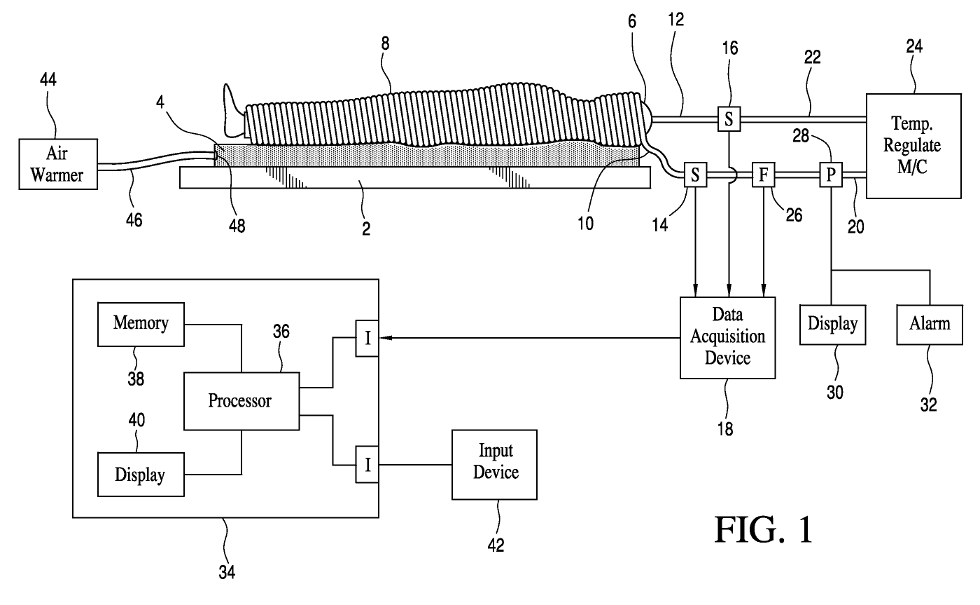

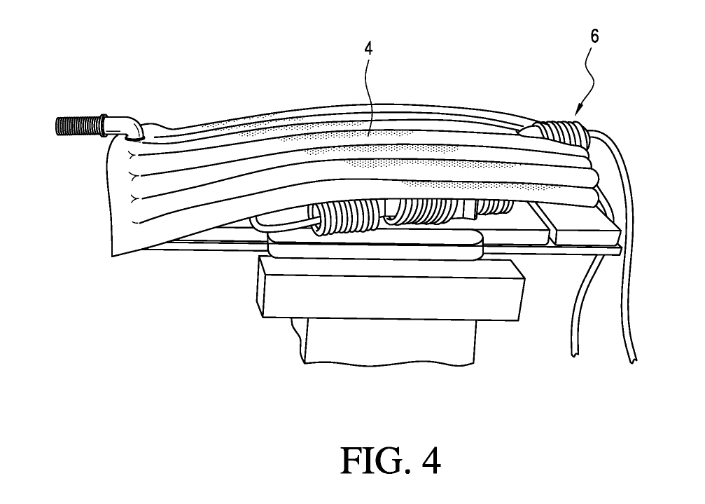

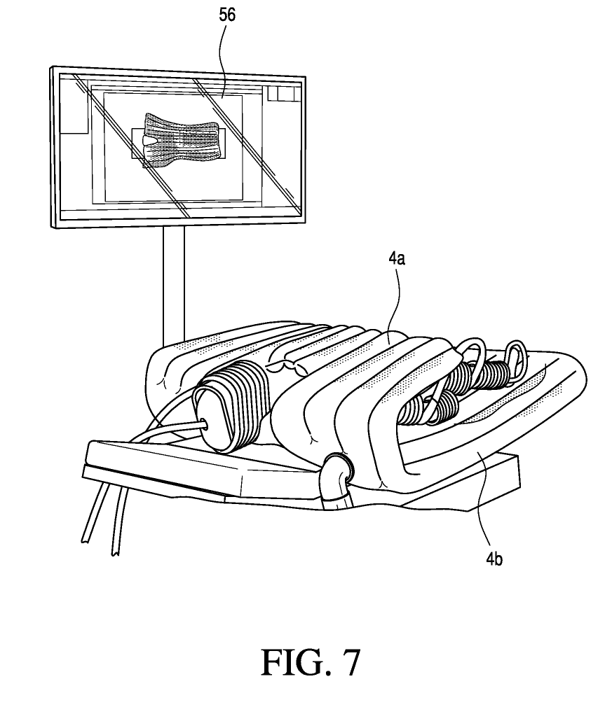

With reference to FIG. 1, an exemplar illustration of an embodiment of the instant invention is shown to include a bed or support 2 onto which a heating device 4 is placed. Heating device 4 for the exemplar illustration is an inflatable convective warming blanket. As is conventionally known, an inflatable convective warming blanket is formed by two air impermeable sheets or layers that are sealed at their respective peripheries with one of the layers having selectively placed air holes or apertures to enable the air input to the blanket to exit. Two convective warming blankets that are marketed by the assignee of the instant invention are shown in FIGS. 4 and 7. It should be noted that even though the exemplar embodiment of the instant invention is described with respect to a convective warming blanket, the heat output efficiency of other types of heating devices such as heating pads and warming blankets (including electric, chemically reactive and recirculating gel) that transfer heat by conduction or other non-conductive heating devices including those using radiating heat output may also be determined using the instant invention.

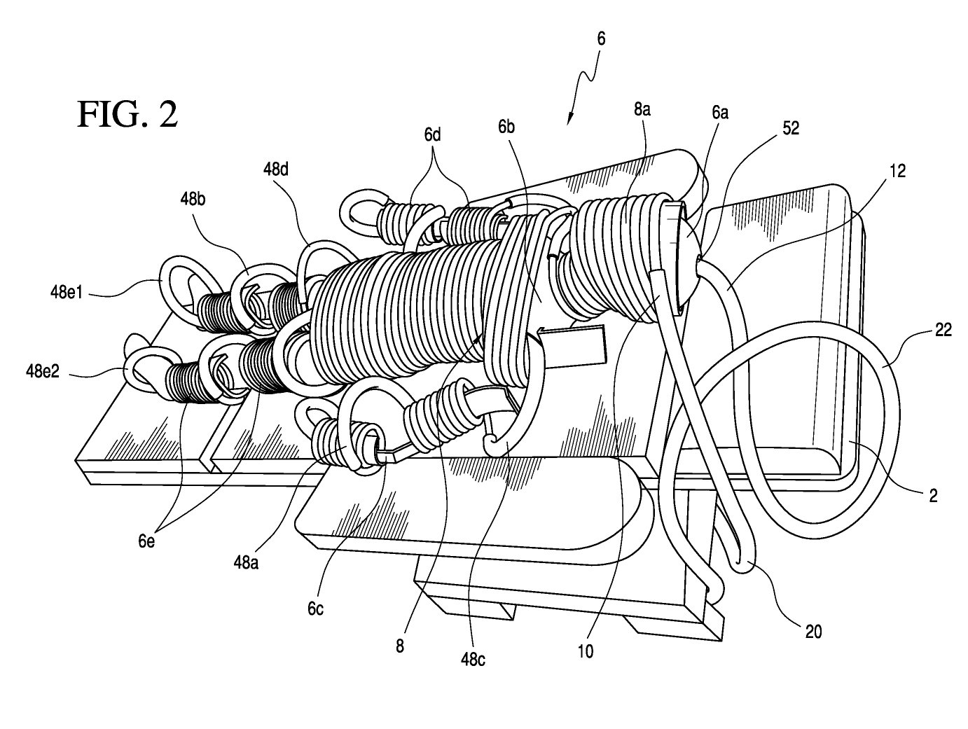

Positioned on top of heating device 4 is a patient or person analog, in the form of a mannequin 6 having a conduit or tube 8 wrapped around its head, torso and other non-moving parts in a coiled fashion. A better illustration of the mannequin with coiled portions of the conduit is shown in FIG. 2, which will be discussed in greater detail infra. For the exemplar embodiment shown in FIG. 1, the mannequin wrapped with the conduit may be considered to be a conduit structure since it is conceivable that a conduit may be shaped, coiled or otherwise, into a structure that resembles a person, or other heat receiving analog forms, without having to be supported by a mannequin.

The conduit structure has an inlet 10 and an outlet 12 to enable a fluid to be input to and output from the conduit structure. Connected in line at inlet 10 is a temperature sensor 14. Another temperature sensor 16 is connected in line at outlet 12. Sensors 14 and 16 measure the respective temperatures of the fluid at the inlet and outlet of the conduit structure. Sensors 14 and 16 each may be an Omega 5TC-TT-T-36-72 T-type thermocouple that measures the temperature of the fluid at the thermocouple site and outputs the measurement as an analog signal to a data acquisition device 18, such as the National Instruments USB-6341X-series data acquisition module. The fluid input to inlet 10 is fed from a tubing 20 that connects inlet 10 to the output of a temperature regulating machine 24. Temperature regulating machine 24 has at its input a tubing 22 connected to inlet 12 of conduit 8 so that a closed fluid circuit system is established between machine 24 and conduit 8. For the embodiment of the instant invention as shown in FIG. 1, with the temperature regulating machine being a chiller, the excess heat picked up by the fluid as the fluid flows through conduit 8 is removed by machine 24, as will be discussed in greater detail infra. Temperature regulating machine 24 may be a ThermoFlex 1400 chiller/circulation machine from the Thermo Scientific Company, a subsidiary of the Fisher Scientific Company of Pittsburgh, Pa.

Provided along the fluid path defined by tubing 20 is a flow meter 26 that monitors the flow rate of the fluid being circulated through the closed system. Flow meter 26 may be an Omega Flowmeter having Model No. FTB603. The measurement taken by flow meter 26 is sent as a signal to data acquisition device 18.

Also provided inline along tubing 20 is a pressure sensor 28 that monitors the pressure of the fluid in the system to ensure that the pressure does not become too high, i.e., exceeds a predetermined value, so as to cause damage to machine 24 and also potentially rupture the closed fluid flow system. The being monitored pressure may be fed to a display and an alarm 32 that outputs an alert or an alarm if the being monitored pressure exceeds the aforenoted predetermined pressure. Pressure sensor 28 may be a Setra Pressure Transducer being marketed by the Setra System Company of Boxborough, Mass. The inline connection of sensors 14 and 16 to inlet 10 and 12, and the inline coupling of flow meter 26 and pressure sensor 28 to tubing 20 are done in a well known conventional manner that does not affect the flow of the fluid along conduit 8 or tubings 20 and 22.

As discussed above, the respective temperature signals from sensors 14 and 16, and the flow rate signal from flow meter 16 are sent to data acquisition device 18. These signals may in turn be forwarded to a work station 34 that includes, among other well known conventional components, a processor 36, a memory 38 and a display 40. An input device 42 such as a keyboard, pointing device or writing tablet may also be connected to work station 34 for inputting instructions or other inputs as is well known. The signals sent from data acquisition device 18 to work station 34 may be recorded and stored in memory 38. Processor 36 is programmed to compute, from the received temperature and flow rate signals, a heating rate that is representative of the amount of heat from the heated air output from the convective warming blanket 4 that is received by the fluid that flows along conduit 8 coiled around mannequin 6, i.e., the conduit structure.

As discussed above, convective warming blanket 4 is made of two air impermeable sheets or layers sealed around their respective peripheries so that the blanket may be inflated by heated air from an air warmer 44 through an air hose 36 connected to an inlet port 4b. The air holes at the one layer of the blanket allows the heated air to escape from the blanket to warm the person or patient, in this instance the person analog in the form of mannequin 6 that is positioned relative to the blanket. For the exemplar embodiment, the layer of the blanket with the air holes is in contact with conduit 8 of the person analog, since mannequin 6 is lying on blanket 4. The air holes that are in contact or in proximity with the coiled tubings of conduit 8 would direct the heated air to the tubings as the fluid flows along conduit 8. As a result, the fluid that flows along conduit 8 is heated. To calculate the amount of heat output from blanket 4 that is received by the person analog, i.e., the heat output efficiency of the blanket, the following equation, which will be discussed in greater detail infra, may be used.

$$Q = ( \frac{dm}{dt} ) * C * Δ T$$

where Q is heating rate,
dm/dt is mass flow rate,
C is heat capacity of the flowing fluid, and
ΔT is the change in fluid temperature between the inlet and outlet.

The LabView software by the National Instruments Corporation of Austin, Tex. may be programmed with the above equation to calculate the heating rate using the measured temperature and flow rate signals.

The person analog in the form of mannequin 6 wrapped with conduit 8 shown in FIG. 2 is positioned onto bed 2. Mannequin 6 is in the shape of a person having a head 6a, a torso 6b, and other non-moving parts including arms 6d and legs 6e. The non-moving parts are joined by movable joints such as 6c at the “elbow” of arm 6d. The head, torso and non-moving parts of the mannequin each are wrapped in a coiled fashion by metal tubings or tubes. For example, the head and the neck of the mannequin are wrapped by coiled tubings 8a. The torso 6b and the other non-moving parts of the mannequin by likewise wrapped by coiled metal tubings. The metal tubings are selected to have good heat transfer characteristics so that heat can readily transfer between the environment and the fluid inside the metal tubings. A good heat conductive metal such as aluminum may be used to make the metal tubings.

The metal tubings are interconnected by flexible PVC plastic tubings or tubes at the movable joints of the mannequin, for example at the “elbow” joint 48a, the “knee” joint 48b, the “shoulder” joint 48c and the “hip” joint 48d. At the “feet” of the mannequin, the respective ends of the metal tubings are connected to corresponding plastic tubings 48e1 and 48e2, which are routed to the interior of the mannequin for connection with other tubings by means of valves, couplings and other means as are conventionally known, so that the interconnected tubings form the one continuous conduit 8 where the fluid flows uninterrupted through the interconnected tubings and output at outlet 12.

Outlet 12 is connected to tubing 22 so that the fluid may be routed to the temperature regulating machine 24. Inlet 10 is connected to tubing 20 wherethrough the fluid from machine 24 is input to conduit 8. With tubings 20 and 22 connecting the conduit structure and machine 24, a closed fluid flow path is effected to enable the fluid to circulate between inlet 10 and outlet 12. Additional fluid may be stored in a reservoir, not shown, in machine 24.

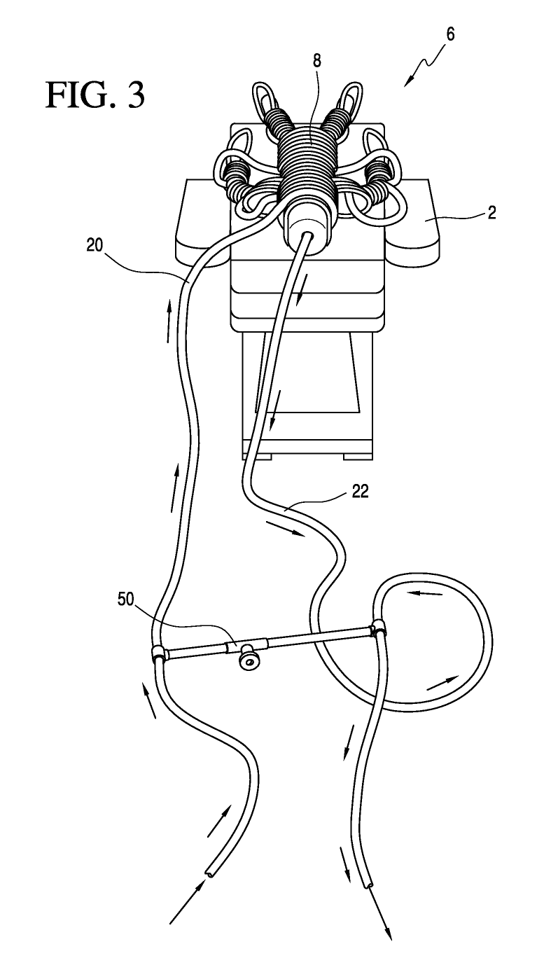

FIG. 3 is another view of the conduit structure shape formed about the mannequin. FIG. 3 shows in particular a bypass valve 50 that may be used to bypass the flow of the fluid in the event that there is a blockage in the person analog conduit due to back pressure built up which may potentially damage the temperature regulating machine 24. Bypass valve 50 works in conjunction with pressure sensor 28 so that, if the internal pressure of the person analog is deemed to rise beyond an acceptable or predetermined value, the bypass valve 50 is turned on to reroute the fluid to flow through the pump of machine 24 without entering into the conduit structure. Valve 50, shown in the highlighted rectangle, may also be used to regulate the inlet pressure to conduit 8 to thereby aid in establishing a specific flow rate for the fluid through the conduit.

FIG. 4 shows the conduit structure, in the form of person analog represented by mannequin 6, being covered by a blanket 4 that is inflated with heated air as discussed above. The layer of blanket 4 that has the air holes is in contact with the conduit structure, so that the person analog receives the heat output from the blanket. FIG. 7 shows a poncho type blanket having a portion 4a covering the front of the torso of the person analog and another portion 4b onto which the person analog lies. The person analog in FIG. 7 therefore receives heat from the blanket at both its front torso as well as its back, as the layer of the convective blanket with the air holes is in contact with both the front of the torso and the back of the person analog.

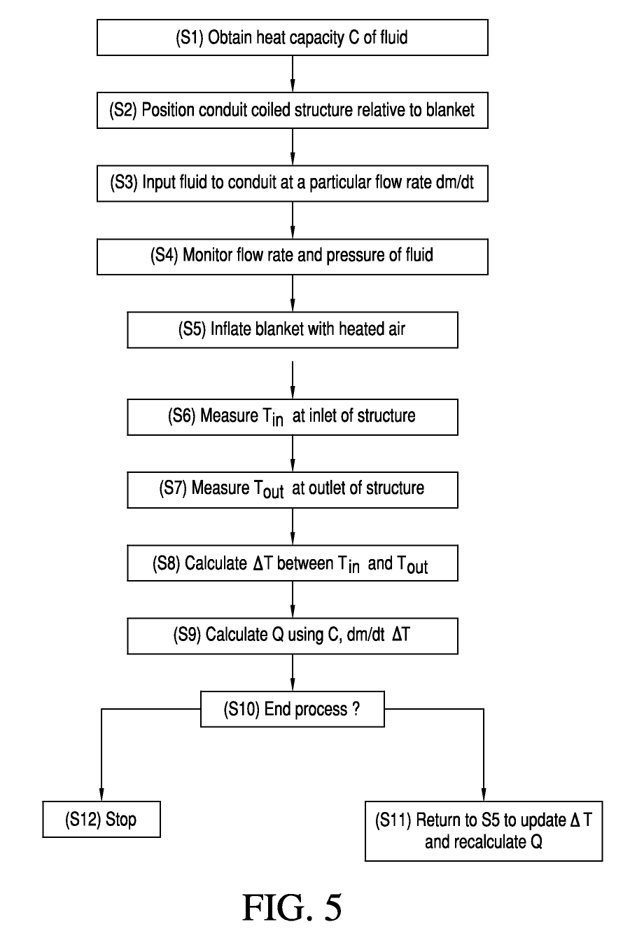

With reference to FIG. 5, the steps for performing the processes of the instant invention method are discussed. Per step S1, the heat capacity C of the fluid to be used is obtained. In the exemplar embodiment at hand, assume the fluid to be used is water at 21° C. From that it can be determined that the water density at 21° C. (69.8° F.) is 998.3 g/L and the specific heat of water at 21° C. is 4.183 j/g° C. With the heat capacity of the fluid known and input into the processor of the workstation 34, the person analog in the form of the coiled conduit structure is positioned relative to the heating device, in this instance the convective warming blanket, per step S2. Per step S3, the fluid is input to conduit 8 of the person analog at a particular flow rate, which may be designated the mass flow rate dm/dt. Per step S4, the flow rate and the pressure of the fluid that flows inside conduit 8 are monitored by flow meter 26 and pressure sensor 28, respectively. Per step S5, with the person analog correctly positioned relative to the blanket, the blanket is inflated by the temperature treated air, in this instance heated air, output from an air warmer. Time is provided to enable the blanket to be inflated fully and the fluid to circulate through the conduit, and also to allow the system to reach an approximately steady state condition wherein the fixed materials in and around the system (including the aluminum tubings, the PVC tubings, the mannequin, and the bed or table or other test surface, the walls of the chiller tank, etc) are not adding or removing heat from the fluid. For example, if the fluid operating temperature is ˜30 C (86° F. above room temperature), then there is some time period after system activation where the fixed materials absorb heat until they reach their steady state temperature. In this case, if measurements were made immediately after system activation, the apparent heat delivered to the fluid would be incorrectly low since the other materials are absorbing some of the heat. This is the primary reason that the fluid used is at room temperature, i.e., 21° C. as noted above, so that the system is as near to the steady state temperature as possible at the start of the process. After the steady state condition is reached, the input temperature Tᵢₙ of the fluid is measured at inlet 10 per step S6, while the output temperature Tₒᵤₜ of the fluid at outlet 12 is measured per step S7. The respective inlet and outlet temperatures measured by sensors 14 and 16, along with the flow rate being measured by flow meter 6, are forwarded to data acquisition device 18, which then sends the collected signals to work station 38. In the work station, which may operate under the above discussed LabView programming environment, processor 36 calculates the difference in temperature ΔT between the input temperature Tᵢₙ and the output temperature Tₒᵤₜ of the fluid, per step S8.

Once the temperature difference ΔT is calculated, per step S9, the amount of heat in watts, i.e., the heating rate Q, that is necessary to heat the fluid that flows through conduit 8 in order to raise the temperature of the fluid from Tᵢₙ to Tₒᵤₜ is calculated using the following equation:

$$Q = ( \frac{dm}{dt} ) * C * Δ T$$

where Q is heating rate,
dm/dt is mass flow rate,
C is heat capacity of the flowing fluid, and
ΔT is the change in fluid temperature between the inlet and outlet.

The mass flow rate is obtained by multiplying the density of the fluid with the volumetric flow rate that is being measured by the flow meter 28. Given that the water density at 21° C. is 998.3 g/L and the specific heat of water at 21° C. is 4.183 J/g° C., the mass flow rate can be obtained by multiplying the measured volumetric flow rate with the density. Thus, by using the above equation, the heating rate that is representative of the amount of heat output from the convective blanket that is received by the conduit structure, i.e., the tubings of conduit 8 coiled about mannequin 6, is calculated to be the following:

Q=Flow[L/min]*(998.3 g/L*1 min/60 s)*4.183 [J/g° C.]*ΔT[° C.]

where the heating rate may also be represented as watts per the following:

Q[Watts]=65.598*Flow[L/min]*ΔT[° C.]

After the calculation of Q per step S9, the process proceeds to step S10 where a determination is made on whether the process is to be terminated. If it is not, then the respective input and output temperatures (Tᵢₙ and Tₒᵤₜ) are measured and the updated temperature signals are sent to the data acquisition device and from there to the processor as described above, so that the heating rate can be recalculated based on the updated temperature difference of the fluid at the input and outlet of the conduit. This is shown in step S11. The instant invention therefore is able to effect a continuous direct measurement of the amount of heat that is being received by the person analog. Putting it differently, the instant invention is able to directly measure the heat output efficacy or efficiency of a heating device, for example a convective warming blanket that outputs heated air to a patient. If the process is determined to end in step S10, the process stops per step S12.

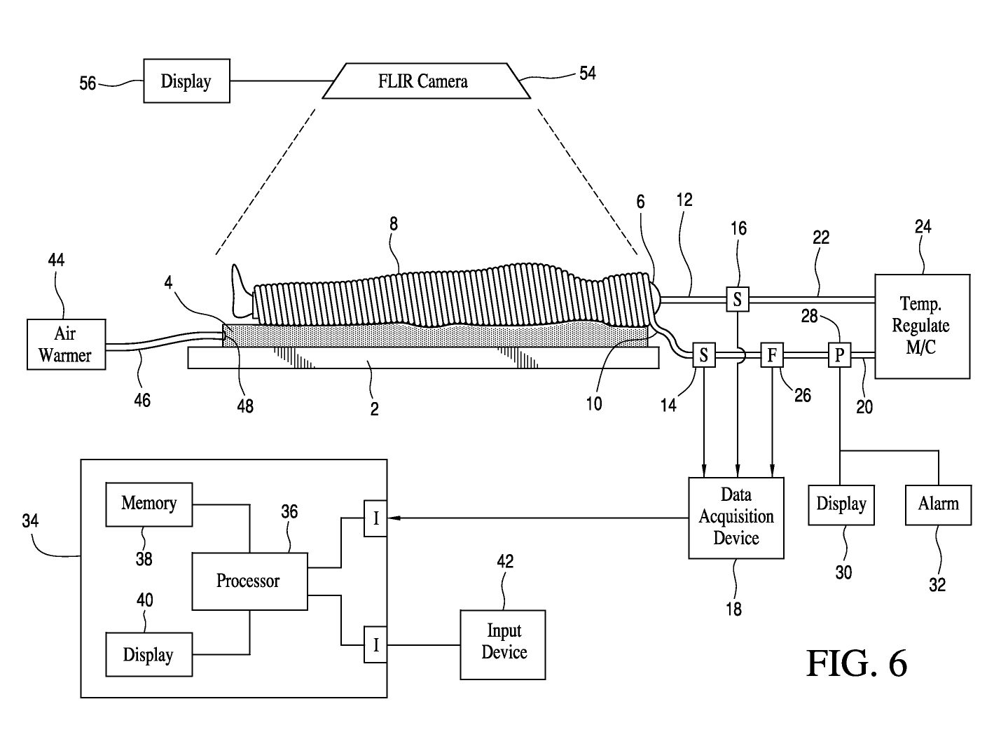

FIG. 6 shows the addition of a forward-looking infrared (FLIR) camera that may be used to determine the heat efficacy of a heating device for example a convective warming blanket, or may be used in conjunction with the inventive method as described above to obtain a redundant check on the heat output efficacy of the blanket. The FLIR camera is made by the FLIR Systems Company of Boston, Mass. In essence, the FLIR camera 54 is positioned above the mannequin, which is on top of blanket 4, to detect the amount of infrared radiation being emitted from a heat source, and from that detection creates a “picture” for video output to display 56.

FIG. 7, as discussed above, shows the person analog being covered by a poncho type convective warming blanket. Also shown in FIG. 7 is the IR image of the person analog and convective blanket captured by FLIR camera 54 being shown on monitor display 52.

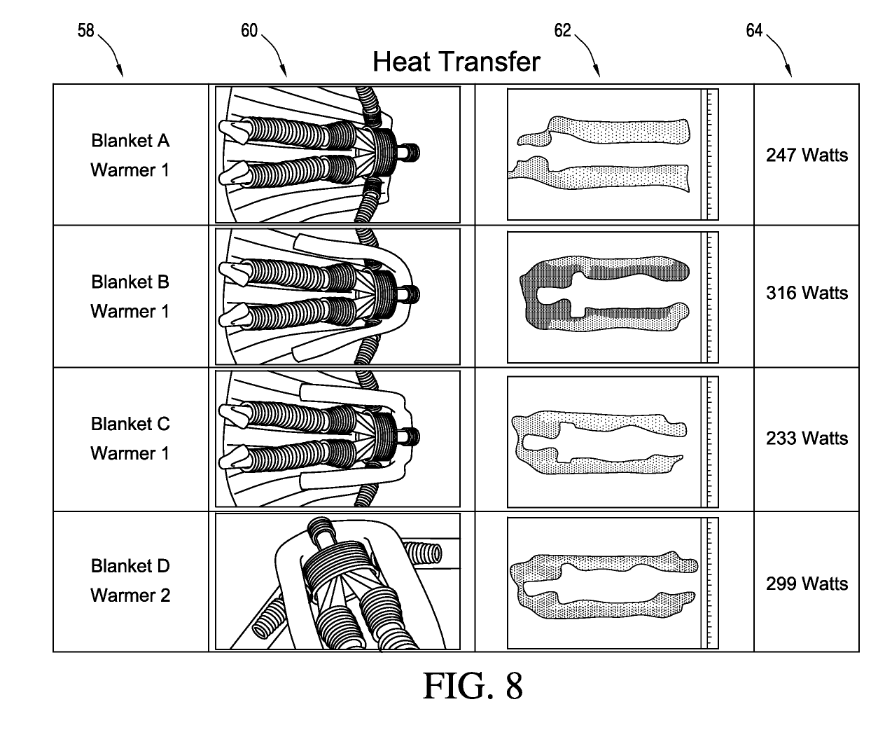

FIG. 8 shows four different blankets A-D heated by different warmers and the respective heating rate, in watts, output by those blankets obtained in accordance with the method of the instant invention. The directly measured heating rates of blankets A-D are compared side-by-side with respective IR images of those blankets captured by the FLIR camera. In particular, column 58 shows blankets A, B and C each inflated by a warmer 1 while blanket D is inflated by a warmer 2. The positioning of the person analog onto the respective blankets are shown in column 60. By means of the inventive method as discussed above, the amount of heat received by the person analog in each of the blankets using the warmers as noted are set forth as watts in column 64. As shown, the heating rate output from blankets A, B and C, each inflated by warmer 1, are 247 watts, 316 watts and 233 watts, respectively. The heating rate of blanket D, inflated by warmer 2, is 299 watts. Column 62 shows the respective IR images of the person analog on the different blankets. Note that the IR image of blanket B confirms that blanket B has the highest output heating rate, as evidenced by the overall brightness of the blanket as shown by the IR image. The FLIR camera captured IR images may therefore act as a complement to confirm the above described inventive method of directly measuring the heating rate of a heating device.

The present invention is subject to many variations, modifications and changes in detail. For example, even though the metal tubings are shown to wrap about the mannequin in a coiled fashion, it may well be that a conduit structure may have a form other than a person analog. For example, a conduit structure may have to be constructed longitudinally along the length of a subject animal such as a dog for use in a veterinary procedure. In which case the heat transfer tubings may be formed in a non-coiled longitudinal configuration. Further, a fluid other than water may also be used, so long as the fluid is flowable and its heat capacity is known. Moreover, instead of plastic tubings to interconnect the metal tubings, other types of flexible tubings including non-plastic tubings may also be used. Furthermore, instead of measuring the heat output efficiency of a heating device, the instant invention may be adapted to monitor and measure the cold output efficiency, or the cooling rate of a cooling device, by replacing the chiller with a warmer and using a fluid that is adapted to flow through the conduit structure without freezing when subjected to the cold output. Thus, it is intended that all matter described throughout this specification and shown in the accompanying drawings be interpreted as illustrative only and not in a limiting sense. Accordingly, it is intended that the instant invention be limited only by the spirit and scope of the hereto attached claims.

1. A method of determining heat output efficiency of a heating device, comprising the steps of:
a) configuring a conduit to from a structure having an inlet and an outlet adapted to be positioned relative to the heating device;
b) circulating a fluid having a heat capacity along the conduit of the structure at a flow rate;
c) measuring the respective temperatures of the fluid at the inlet and the outlet of the structure; and
d) calculating a heating rate that is representative of the amount of heat received by the structure from the heating device by using the flow rate and the measured respective inlet and outlet temperatures of the fluid.

2. The method of claim 1, further comprising the steps:
removing heat from the fluid output from the outlet to maintain the fluid at a given temperature;
returning the fluid with the given temperature to the inlet for circulation along the conduit;
continue to measure the respective temperatures of the fluid circulating along the conduit at the inlet and outlet of the structure to obtain updated temperature differences of the fluid at the inlet and outlet of the structure; and
recalculating the heating rate by using the flow rate and the updated temperature differences of the fluid.

3. The method of claim 1, wherein step (d) comprises using the formula
$$Q = ( \frac{dm}{dt} ) * C * Δ T$$
where Q is heating rate,
dm/dt is mass flow rate,
C is heat capacity of the flowing fluid, and
ΔT is the change in fluid temperature between the inlet and outlet.

4. The method of claim 1, wherein the structure is shaped in the form of a person adapted to lie on top of the heating device, covered by the heating device, or lie on top of one portion and covered by another portion of the heating device.

5. The method of claim 4, wherein the heating device comprises an inflatable convective blanket formed by two air impermeable layers having apertures at one of the layers that faces the structure for outputting heated air towards the structure.

6. The method of claim 1, wherein the conduit comprises metal tubings having a good heat transfer characteristic and flexible plastic tubings, wherein step (a) further comprises the steps of;
providing a body adapted to be positioned relative to the heating device having an exterior in the shape of a person having a torso and non-moving body parts connected by moving joints and an interior;
wrapping the exterior of the torso and the non-moving parts of the body with the metal tubings;
interconnecting the metal tubings with the plastic tubings at the movable joints of the body and within the interior of the body so that the conduit form fits about the exterior of the body to effect the structure and to establish a closed fluid flow path between the inlet and the outlet.

7. The method of claim 2, wherein the removing heat step comprises using a chiller to remove the heat collected by the fluid output from the conduit in excess of the temperature of the fluid input to the conduit.

8. The method of claim 1, further comprising the steps of:
providing a flow meter to monitor the flow rate of the fluid along the conduit;
maintaining the flow of the fluid at a desired flow rate;
providing a pressure sensor to monitor the pressure of the fluid in the conduit; and
outputting an alarm if the monitored pressure of the fluid in the conduit is higher than a predetermined value.

9. The method of claim 1, wherein the fluid is water and wherein the heating rate may be displayed as a unit of watts to represent the amount of heat received by the structure.

10. The method of claim 1, further comprising the step of:
positioning a forward looking infrared (FLIR) camera relative to the structure to capture a reflected apparent temperature IR image of the structure and the heating device.

11. Apparatus for determining heat output efficiency of a heating device, comprising:
a structure formed by a conduit having an inlet and an outlet positioned relative to the heating device;
a pump for circulating a fluid having a heat capacity along the conduit at a flow rate;
respective sensors at the inlet and the outlet of the structure for measuring corresponding temperatures of the fluid at the inlet and the outlet; and
processor means for calculating a heating rate representative of the amount of heat received by the structure from the heating device by using the flow rate and the measured inlet and outlet temperatures of the fluid

12. The apparatus of claim 11, further comprising:
a temperature regulating device to remove heat from the fluid output from the outlet to maintain the fluid at a given temperature;
a pump to return the fluid with the given temperature to the inlet for circulation along the conduit;
wherein the respective sensors continue to measure the corresponding temperatures of the fluid at the inlet and outlet as the fluid is circulated along the conduit to obtain updated temperatures of the fluid at the inlet and outlet;
wherein the processor means recalculates the heating rate by using the flow rate and the updated temperatures of the fluid at the inlet and outlet.

13. The apparatus of claim 11, wherein the processor means calculates the heating rate by using the formula
$$Q = ( \frac{dm}{dt} ) * C * Δ T$$
where Q is heating rate,
dm/dt is mass flow rate,
C is heat capacity of the flowing fluid, and
ΔT is the change in fluid temperature between the inlet and outlet.

14. The apparatus of claim 11, wherein the heating device comprises an inflatable convective blanket formed by two air impermeable layers having apertures at one of the layers that faces the structure for outputting heated air towards the structure.

15. The apparatus of claim 11, wherein the structure comprises the conduit winding about various parts of a mannequin, the conduit wound mannequin adapted to lie on top of the heating device, covered by the heating device, or lie on top of one portion and covered by another portion of the heating device.

16. The apparatus of claim 11, further comprising a temperature regulating device to remove the heat collected by the fluid as the fluid flows from the inlet to the outlet in excess of the temperature of the fluid input to the conduit.

17. The apparatus of claim 11, further comprising:
a flow meter to monitor the flow rate of the fluid flowing along the conduit; and
a pressure sensor to monitor the pressure of the fluid in the conduit;
wherein an alarm signal is output if the monitored pressure of the fluid in the conduit is higher than a predetermined value.

18. The apparatus of claim 11, wherein the conduit comprises metal tubings and flexible plastic tubings, the metal tubings adapted to readily transfer heat between the fluid in the tubings and the environment; and
wherein the structure comprises a body having an exterior in the shape of a person having a torso and non-moving parts connected by moving joints and an interior, the exterior of the torso and other non-moving parts of the body wrapped with the metal tubings; and
wherein the metal tubings are interconnected with the plastic tubings at the movable joints of the body and within the interior of the body so that the conduit forms the structure at the exterior of the body and establishes a closed fluid flow path between the inlet and the outlet.

19. Apparatus for determining heat output efficiency of a convective blanket formed by two air impermeable layers inflatable by heated air and having selectively placed apertures at one of its layers, comprising:
a structure formed by a conduit having an inlet and an outlet positioned relative to the blanket such that at least some of the apertures face the structure for outputting heated air towards the structure;
a pump for circulating a fluid along the conduit at a particular flow rate;
respective sensors at the inlet and the outlet of the structure for measuring the temperature of the fluid at the inlet and the temperature of the fluid at the outlet; and
a processor for calculating a heating rate representative of the amount of heat received by the structure from the blanket by using the flow rate and the measured inlet and outlet temperatures of the fluid.

20. The apparatus of claim 19, further comprising:
a temperature regulating device to remove an amount of heat from the fluid output from the outlet that is in excess of the heat in the fluid input to the inlet, the excess heat removed fluid being returned to the inlet for circulation along the conduit;
wherein the sensors continue to measure the respective temperatures of the fluid at the inlet and outlet as the fluid is circulated along the conduit to obtain updated temperatures of the fluid at the inlet and outlet;
wherein the processor recalculates the heating rate by using the updated temperatures of the fluid at the inlet and outlet.

21. The apparatus of claim 19, wherein the processor calculates the heating rate by using the formula
$$Q = ( \frac{dm}{dt} ) * C * Δ T$$
where Q is heating rate,
dm/dt is mass flow rate,
C is heat capacity of the flowing fluid, and
ΔT is the change in fluid temperature between the inlet and outlet.

22. The apparatus of claim 19, wherein the structure comprises a mannequin including a torso and non-moving parts having an exterior surface, the non-moving parts joined by movable joints, the exterior surface of the torso and non-moving parts wrapped by coiled metal tubings adapted to readily transfer heat between the fluid in the tubings and the environment, the metal tubings interconnected to flexible plastic tubings at the movable joints and within interior of the mannequin so that the fluid flows uninterrupted along the conduit between the inlet and the outlet.

23. A person analog adapted to collect heat directed thereto, comprising:
a body having an interior and an exterior shaped to have a head, torso and non-moving body parts connected by moving joints, the exterior of the head, torso and the non-moving parts being wrapped with heat conductive metal tubings interconnected with flexible plastic tubings at the movable joints of the body and within the interior of the body, the interconnected metal and plastic tubings forming a continuous conduit to establish a closed fluid flow path between an inlet and an outlet of the conduit through which a fluid adapted to collect the heat directed to the person analog flows.

24. The person analog of claim 23, wherein the inlet and outlet are connected to output and input connectors, respectively, of a pump mechanism of a temperature regulation device so that the fluid may be circulated between the person analog and the device where the collected heat is removed from the fluid.
